(19) United States
(12) Patent Application Publication
(10) Pub. No.: US 2022/0193175 A1
(43) Pub. Date: Jun. 23, 2022

(54) FUNCTIONAL TEA CONTAINING BENEFICIAL INGREDIENTS FOR THE HUMAN BODY

(71) Applicant: MEDIPRESSO,. CO. LTD, Daejeon (KR)

(72) Inventors: Ha Seob KIM, Daejeon (KR); Hae Sung LEE, Daejeon (KR)

(21) Appl. No.: 17/135,152

(22) Filed: Dec. 28, 2020

(30) Foreign Application Priority Data
Dec. 21, 2020 (KR) 10-2020-0179399

(51) Int. Cl. A61K 36/899; A61K 36/484

(52) U.S. Cl. CPC A61K 36/899; A61K 36/484

(57) ABSTRACT

The present disclosure relates to a functional tea containing beneficial ingredients for the human body.

### CROSS-REFERENCE TO RELATED APPLICATION

This application claims priority to Korean Patent Application No. 10-2020-0179399 filed on Dec. 21, 2020 and all the benefits accruing therefrom under 35 U.S.C. § 119, the contents of which are incorporated by reference in their entirety.

### BACKGROUND

The present disclosure relates to a functional tea which contains a large amount of beneficial ingredients for the human body.

Today, modern people are under much mental stress due to rapid changes in life culture and environment and are suffering from chronic fatigue, a decrease in physical strength, and various adult diseases due to excessive drinking and physical activity.

In particular, many people at work consume coffee containing caffeine to relieve fatigue or mental stress. However, coffee is known to also cause side effects such as sleep disorder, insomnia, gastrointestinal disorder, dehydration due to diuretic action, a decrease in bone density and subsequent arthritis, an increase in blood pressure, leg tremors, persistent convulsion, etc.

Therefore, in recent years, the demand for functional beverages that can replace such coffee is increasing, and to meet such a need of consumers, various beverages and teas beneficial for the body have been continuously developed.

However, most of the teas currently on sales in the market are those which are prepared by processing green tea, black tea, etc., with already known functions, and their diversity is not sufficient to meet the various tastes of modern people and moreover when considering even their functionalities.

In this regard, recently, the demand for tea containing herbal ingredients that are beneficial for the human body is on the increase, however, some consumers are not favored for the peculiar flavors derived from herbal ingredients.

Accordingly, the present inventors have made extensive efforts to develop a functional tea with excellent taste and flavor although the functional tea contains a large amount of beneficial ingredients for the human body by containing herbal ingredients. As a result, they have succeeded in developing a functional tea containing a large amount of beneficial ingredients for the human body while having high preference without a peculiar sense of rejection by combining various herbal ingredients, fruit ingredients, natural flavors, and fruit seed ingredients.

Throughout this specification, many papers and patent documents are referenced and their citations are indicated. The disclosure contents of the cited papers and patent documents are incorporated by reference in this specification in their entirety such that the level of the technical field to which the present disclosure belongs and the contents of the present disclosure are more clearly explained.

### SUMMARY

The present inventors have made extensive efforts to develop a functional tea with excellent taste and flavor while containing a large amount of beneficial ingredients for the human body by containing herbal ingredients.

Accordingly, the present disclosure has solved the conventional drawback in the functional tea by using a combination of raw materials of a tea containing herbal ingredients, and the present disclosure is an invention which relates to a tea which can be selected considering the health of consumers and simultaneously having a taste and a flavor while containing beneficial ingredients for the human body.

The present disclosure relates to a functional tea in consideration of consumers' health and preferences and a preparation method thereof.

In accordance with an exemplary embodiment of the present invention, a functional tea contains:

dried tea leaves containing one or more ingredients selected from dry rooibos, dry lotus leaves, dry chamomile, dry hibiscus, dry lemongrass, and dry stevia;

one or more herbal ingredients selected from Puerariae radix, Glycyrrhiza uralensis, Cinnamomum japonicum, Lycium chinese, Angelica gigas, Zizyphus jujuba, Paeonia lactiflora, Rehmanniae radix, Schisandra chinensis, longan, Citri reticulatae Pericarpium, Nidium officinale Makino, Astragalus membranaceus, Houttuynia cordata; and

fruit ingredients.

The herbal ingredients contained in the functional tea of the present disclosure are herbal ingredients that can exhibit a detox effect, and their main function is to discharge waste products from the body of consumers through the functional tea of the present disclosure. This is called teatox (a compound word of tea and detox).

In addition to the waste discharge function described above, by containing herbal ingredients, the functional tea of the present disclosure can have beneficial effects for the human body such as preventing colds, increasing immunity, easing insomnia, lowering blood pressure, skin care, relaxation of nerves, anti-oxidation, lowering blood sugar levels, and preventing aging.

In particular, the herbal ingredients are characterized in that they are contained in an amount of 10-20 mass %, and preferably 13-17 mass % with respect to the total weight of the functional tea of the present disclosure. When the content of the herbal ingredients is lower than the above range, the effect due to herbal ingredients cannot be exhibited, whereas when the content of the herbal ingredients is higher than the above range, the flavor of herbal ingredients is too strong to satisfy consumers' preferences. Therefore, it is very important to match the content of herbal ingredients within the above range (see FIG. 1).

According to an exemplary embodiment of the present disclosure, the fruit ingredients are characterized in that they are one or more fruit ingredients selected from apple, tangerine, lemon, peach, pear, and Schisandra chinensis. In particular, the fruit ingredients were discovered through numerous trials and errors, and the inventors of the present disclosure have found a combination of fruit ingredients and herbal ingredients which exhibits a taste and a flavor without a rejection.

Fruit ingredients other than the above fruit ingredients are not preferred due to the taste and flavor that cause a feeling of rejection when they are combined with herbal ingredients.

In accordance with an exemplary embodiment of the present invention, it is preferred that fruit ingredients be contained in an amount of 1-3 mass % with respect to the total weight of the raw materials of the functional tea of the present disclosure.

In accordance with an exemplary embodiment of the present invention, the functional tea of the present disclosure is characterized in that the functional tea further contains a natural flavor. A mixture in which tea leaves, herbal ingredients, fruit ingredients, and natural flavors are mixed is the raw material for a functional tea of the present disclosure. In the raw material of the functional tea, the weight excluding the herbal ingredients, fruit ingredients, and natural flavors can be appropriately adjusted by the weight of the tea leaves.

By further containing the natural flavors, the functional tea of the present disclosure can have more excellent flavor and aroma while holding the unique flavors of herbal ingredients. The natural flavors may be selected from a natural apple flavor, a natural peach flavor, a natural cranberry flavor, a natural plum flavor, a natural tangerine flavor, and a natural cardamon flavor.

In particular, it is preferred that natural flavors be contained in an amount of 1-3 mass % with respect to the total weight of the raw materials of the functional tea of the present disclosure. When the natural flavors are contained outside the above range, it is not preferred because the flavor and aroma due to the natural flavors may not be felt at all, or the flavor due to natural flavors becomes too strong.

Moreover, the functional tea of the present disclosure contains a fruit seed component in a powder form, such as grape seeds or watermelon seeds, and thus can provide a functional tea containing a linolenic acid component that lowers cholesterol levels or a polyphenol component that prevents cardiovascular disease.

In particular, the powder of grape seeds or watermelon seeds is characterized in that the powder is further contained in an amount of 1-5 mass % with respect to the total weight of the raw materials of the functional tea of the present disclosure.

Meanwhile, the functional tea of the present disclosure is characterized in that the functional tea is prepared in the form of a capsule or tea bag, and when the functional tea is prepared in the form of a tea bag, the mesh size of the tea bag is characterized in that the tea bag is prepared in a size of 10-20 μm in consideration of the size of the fruit seed powder. Since the fruit seeds are prepared in powder, when the mesh size of the tea bag is larger than the above range, the fruit seed powder component may be released out of the tea bag, and in this case, it is not desirable because consumers may have a feeling of rejection when drinking the same.

Meanwhile, the functional tea of the present disclosure is characterized in that the functional tea contains a polyphenol component in an amount of 100-200 mg of gallic acid/mL when the functional tea is brewed.

In addition, the functional tea of the present disclosure is characterized in that the functional tea contains 200-300 mg of linolenic acid when the functional tea is brewed.

Meanwhile, the functional tea of the present disclosure is characterized in that when the functional tea is brewed, the functional tea has a brix in the range of 0.1 to 1, and preferably in the range of 0.1 to 0.8, and additionally, is characterized in that the functional tea has a pH in the range of 2.0 to 6.0.

The ranges of brix and pH are the values discovered by the inventors of the present disclosure through numerous trials and errors, and it was confirmed that general consumers' preferences for the functional tea increased when the brix and pH are retained within the above ranges.

Meanwhile, in accordance with another exemplary embodiment of the present invention, a method for preparing a functional tea includes:

(a) drying a mixture, in which tea leaves, herbal ingredients, and fruit ingredients are mixed, at 30-50° C. for 4-8 hours;

(b) roasting the mixed mixture at 100-200° C. for 5-30 minutes;

(c) pulverizing the mixture which underwent the step of roasting to a size of 0.1-0.5 cm; and

(d) adding grape seeds powder, which is pulverized to a size of 50-100 μm, to the pulverized mixture; and

(e) sterilizing the resultant with ultraviolet (UV) rays in the range of 100-700 nm.

### BRIEF DESCRIPTION OF THE DRAWINGS

FIG. 1 is a result of a flavor test result of a functional tea prepared according to an exemplary embodiment of the present disclosure and a functional tea prepared by the preparation method of Comparative Example 1. It was confirmed that the flavor feeling felt by consumers decreased sharply as the herbal ingredients exceeded 20 mass % of that of the raw materials of the functional tea.

FIG. 2 is a result of a flavor test result of a functional tea prepared according to an exemplary embodiment of the present disclosure and a functional tea prepared by the preparation method of Comparative Example 2. In the case of a functional tea which did not contain a natural flavor, it was confirmed that the flavor feeling was lower than that of the functional tea containing a natural flavor.

FIG. 3 is a result of a flavor test result of a functional tea prepared according to an exemplary embodiment of the present disclosure and a functional tea prepared by the preparation method of Comparative Example 3. In the case of a functional tea which did not contain a fruit ingredient, it was confirmed that the flavor feeling was lower than that of the functional tea containing a fruit ingredient.

### DETAILED DESCRIPTION OF EMBODIMENTS

Examples of the functional tea according to the present disclosure can be applied in various ways, and they should not be construed as being limited to the embodiments set forth herein, but it should be understood to include all of the changes, equivalents, or substitutes included in the spirit and scope of the present disclosure.

### EXAMPLES

Throughout this specification, “%” used to indicate the concentration of a specific substance is in mass % unless otherwise stated.

### Example 1. Preparation of Functional Tea Containing Hibiscus

1. Preparation Step

Raw materials of a functional tea were prepared by mixing 30 mass % of hibiscus leaves, 30 mass % of lemongrass leaves, and 20 mass % of stevia leaves as tea leaves; 15 mass % of Glycyrrhiza uralensis as herbal ingredients; and 2 mass % of dried tangerine, 2 mass % of a natural peach flavor, and 1 mass % of a natural cranberry flavor. In addition, the raw materials of the functional tea were dried at 40° C. for 6 hours. Thereafter, the roasting process was performed and naturally cooled to room temperature to prepare raw materials of a roasting-treated functional tea. In particular, the roasting process was performed at 150° C. for 10 minutes.

2. Pulverization Step

The raw materials of a functional tea were pulverized to a size of 0.2-0.3 cm using a cutter and a grinder. Approximately 90% of the pulverized fine powder generated after the pulverization was removed.

3. Mixing and Sterilization

The raw materials, which underwent the pulverization, and the grape seed powder, which were pulverized into a size of 50-100 μm, were put into a ribbon mixer, and then mixed with the pulverized raw materials. The raw materials were sterilized through UV, etc. installed inside while mixing the raw materials in the ribbon mixer. In particular, the sterilization was performed by adjusting UV rays at nanometer levels.

4. Filling Capsules

After filling the raw materials, which are mixed into the capsules for packaging, using a device for filling capsules, and the capsules containing the raw materials were sealed with a non-woven filter and an aluminum sealing paper.

5. Preparation of Functional Tea

The filled functional tea capsules were brewed in hot water at 90° C. for 3 minutes and thereby a finished functional tea was prepared.

Measurement of Polyphenol Content

The total phenolic compound content was measured by applying the Folin-Denies method (Amerinem & Ough 1958). The lyophilized sample was extracted with methanol at a concentration of 1 mg/mL, and 50 μL of 50% Folin-Ciocalten reagent was added to 100 μL of the supernatant and mixed. After allowing the mixture to stand at room temperature for 3 minutes, 150 μL of a 2% Na₂CO₃ solution was added thereto and reacted for 30 minutes. After completion of the reaction, the absorbance was measured at 700 nm using an ELISA microplate reader (Infinite® 200 PRO, TECAN Group Ltd., Switzerland), and the content was obtained from the drafted standard curve. In particular, gallic acid (Sigma-Aldrich, USA) was used as the reference material and the analysis was performed in the same manner as in the sample, and the total phenolic compound content was obtained from the obtained standard curve.

### Example 2

A functional tea was prepared in the same manner as in Example 1 except that chamomile leaves were added instead of hibiscus leaves and lemongrass leaves, and Citri reticulatae Pericarpium was added instead of Glycyrrhiza uralensis.

### Comparative Example 1

A functional tea was prepared in the same manner as in Example 1 except that herbal ingredients were contained in an amount of 30 mass %.

### Comparative Example 2

A functional tea was prepared in the same manner as in Example 1 except that no natural flavor was added.

### Comparative Example 3

A functional tea was prepared in the same manner as in Example 1 except that no fruit ingredient was added.

### Experimental Example

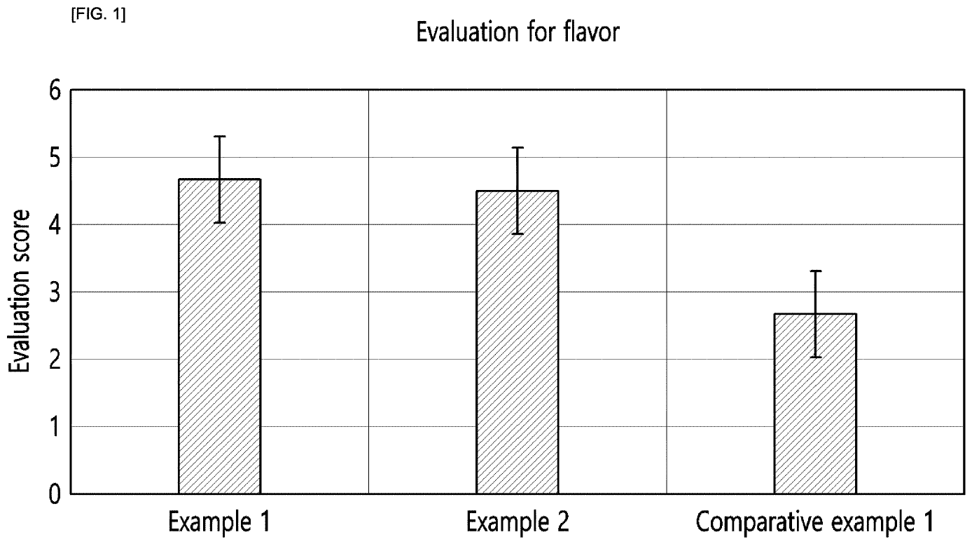

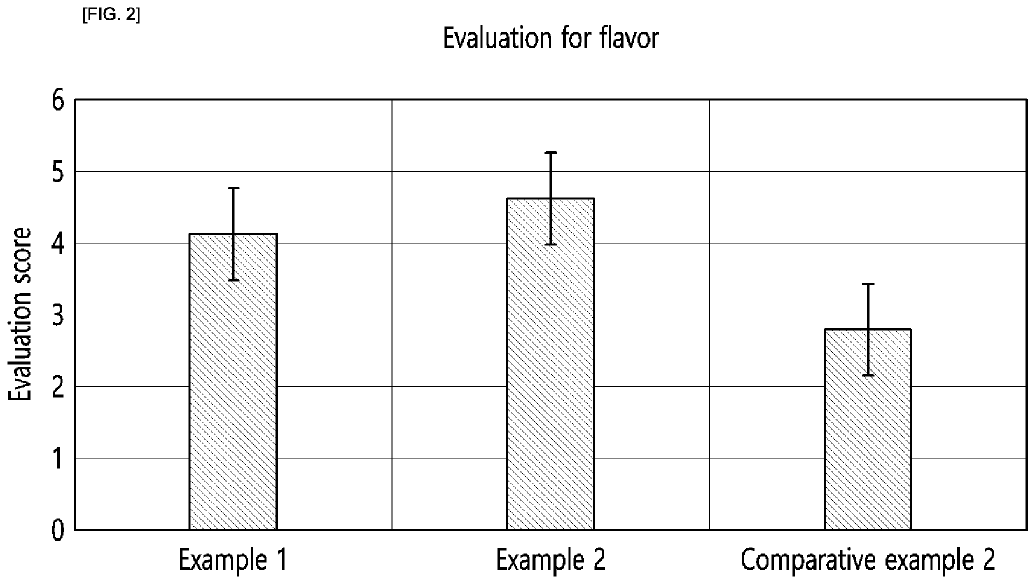

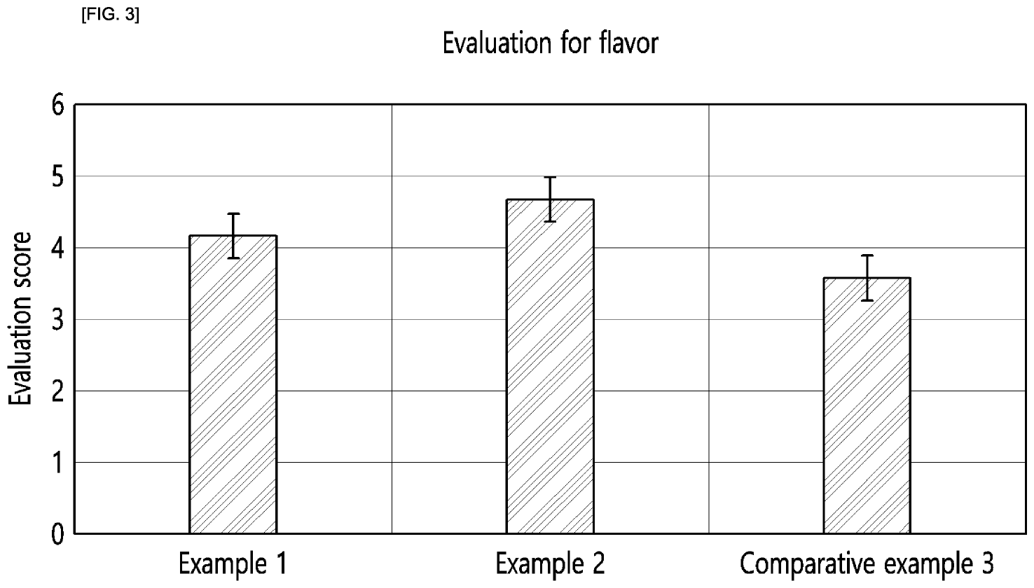

A sensory test for flavor was performed using the functional teas from Examples 1 and 2 and Comparative Examples 1, 2, and 3. The sensory test was performed in the vicinity of Gasan Digital Station in Seoul (Korea). The sensory test was performed by allowing 50 people of various ages passing by in the vicinity of Gasan Digital Complex Station in Seoul (Korea) to taste functional teas. In particular, scores were calculated using a 5-point test for the following items, and the results are shown in FIGS. 1 to 3.

Evaluation Standard

1. The taste and the flavor are very poor.

2. The taste and the flavor are not good.

3. The taste and the flavor are average.

4. The taste and the flavor are good.

5. The taste and the flavor are very good.

As a result of the above test, most people, regardless of gender and age, evaluated the functional teas of the present disclosure as having a very good taste and a very good flavor.

### Advantageous Effects of the Invention

According to the present disclosure, a functional tea that can be enjoyed by people of all ages can be provided by using ingredients that generate various flavors and aromas, although the functional tea contains herbal ingredients.

According to the present disclosure, a functional tea that have beneficial effects for the human body, such as preventing colds, increasing immunity, easing insomnia, lowering blood pressure, skin care, relaxation of nerves, anti-oxidation, lowering blood sugar levels, and preventing aging by containing herbal ingredients, in addition to a waste discharge function (i.e., a detox effect) by using combinations of raw materials of a tea with specific properties, can be provided.

Although the functional tea containing beneficial ingredients for the human body has been described with reference to the specific embodiments, it is not limited thereto. Therefore, it will be readily understood by those skilled in the art that various modifications and changes can be made thereto without departing from the spirit and scope of the present invention defined by the appended claims.

What is claimed is:

1. A functional tea comprising:
dried tea leaves comprising one or more ingredients selected from dry rooibos, dry lotus leaves, dry chamomile, dry hibiscus, dry lemongrass, and dry stevia;
one or more herbal ingredients selected from Puerariae radix, Glycyrrhiza uralensis, Cinnamomum japonicum, Lycium chinese, Angelica gigas, Zizyphus jujuba, Paeonia lactiflora, Rehmanniae radix, Schisandra chinensis, longan, Citri reticulatae Pericarpium, Nidium officinale Makino, Astragalus membranaceus, Houttuynia cordata; and
one or more fruit ingredients selected from apple, tangerine, lemon, peach, pear, and Schisandra chinensis.

2. The functional tea of claim 1, wherein the herbal ingredients are contained in an amount of 10-20 mass % with respect to the total weight of the functional tea.

3. The functional tea of claim 2, further comprising one or more natural flavors selected from a natural apple flavor, a natural peach flavor, a natural cranberry flavor, a natural plum flavor, a natural tangerine flavor, and a natural cardamon flavor.

4. The functional tea of claim 3, wherein the natural flavors are contained in an amount of 1-3 mass % with respect to the total weight of the raw materials of the functional tea.

5. The functional tea of claim 4, wherein when the functional tea is brewed with water at 90-100° C. for 2-3 minutes, the resultant has a brix of 0.1 to 1 and has a pH in the range of 2.0 to 6.0.

6. A method for preparing a functional tea, comprising:
(a) drying a mixture, in which tea leaves, herbal ingredients, fruit ingredients, and natural flavors are mixed, at 30-50° C. for 4-8 hours;
(b) roasting the mixed mixture at 100-200° C. for 5-30 minutes;
(c) pulverizing the mixture which underwent the step of roasting to a size of 0.1-0.5 cm; and
(d) adding grape seeds powder, which is pulverized to a size of 50-100 μm, to the pulverized mixture; and
(e) sterilizing the resultant with ultraviolet (UV) rays in the range of 100-700 nm.
